(19) United States
(12) Patent Application Publication
(10) Pub. No.: US 2026/0174587 A1
(43) Pub. Date: Jun. 25, 2026

(54) OSTOMY CUSTOM SKIN BARRIER METHOD AND SYSTEM

(71) Applicants: Jason Jit-Sun Tan, West Leederville (AU); Kwang Guan Tay, West Leederville (AU)

(72) Inventors: Jason Jit-Sun Tan, West Leederville (AU); Kwang Guan Tay, West Leederville (AU)

(21) Appl. No.: 19/128,925

(22) PCT Filed: Nov. 8, 2023
(86) PCT No.: PCT/AU2023/051125

§ 371 Date: May 9, 2025

(30) Foreign Application Priority Data
Nov. 9, 2022 (AU) 2022903353

(51) Int. Cl. A61F 5/445; G05B 19/4093

(52) U.S. Cl. CPC A61F 5/445; G05B 19/40931; G05B 2219/32036

(57) ABSTRACT

Provided is a method 30 for producing a custom ostomy skin barrier 12, said method 30 comprising the steps of providing 32 a scanner 16 whereby a user is able to scan a torsal peristomal area 18, said scanner 16 configured to generate 34 a data image of the scanned peristomal area 18, and transmitting 36, by the scanner 16, the data image to a skin barrier production system 22. The method 30 also includes the step of analysing 38, by the production system 22, the received data image in order to produce 40 a skin barrier template whereby a custom ostomy skin barrier 12 corresponding with a stoma 20 in the scanned peristomal area 18 is producible.

### TECHNICAL FIELD

This invention relates broadly to ostomy pouching systems, and more particularly to a method and associated system for a custom ostomy skin barrier.

### BACKGROUND ART

The following discussion of the background art is intended to facilitate an understanding of the present invention only. The discussion is not an acknowledgement or admission that any of the material referred to is or was part of the common general knowledge as at the priority date of the application.

An ostomy is a surgery that makes a temporary or permanent opening in the skin called a stoma. A stoma is a pathway from an internal organ to the outside of the abdomen. Examples include a colostomy, which originates in the large intestine, and an ileostomy, which originates in the small intestine. A stoma is often irregular and of varying sizes.

An ostomy pouching system is a prosthetic medical device that provides a means for the collection of waste from a surgically diverted biological system (e.g. colon, ileum, bladder, etc.). Pouching systems usually consist of a collection pouch and a barrier on the skin, which connects with the stoma itself, being the part of the body that has been diverted to the skin. The pouching system generally allows the stoma to drain into a sealed collection pouch, while protecting the surrounding skin from contamination. These systems are used to maintain independence, so that a wearer can continue to lead an unrestricted lifestyle.

Ostomy barriers sit on the skin and separate the ostomy pouch from the internal conduit. These skin barriers, also called flanges, wafers, or baseplates, are manufactured in different sizes to accommodate a person's particular anatomy. The internal opening of the skin barrier must be the correct size to accommodate the individual's stoma while protecting the skin from contact with waste. The methods for sizing this opening vary depending on the type of wafer/baseplate; some pre-cut sizes are available; and some users customize the opening using scissors.

An ostomy pouching system generally comes in two types, namely one-piece or two-piece systems. Examples of these systems are shown in FIGS. 2 and 3. The type of system used generally varies between individuals and is often based on the medical reason, personal preference and lifestyle. As shown, a one-piece system consists of a skin barrier/wafer and pouch joined together as a single unit. A two-piece system consists of a separate skin barrier/wafer that can be placed on the skin with the collection pouch that is removable from the skin barrier.

One-piece systems generally provide greater simplicity than two-piece systems, but require changing the entire unit, including skin barrier, when the pouch is changed. Two-piece systems allow changing pouches while leaving the barrier/wafer attached to the skin, where the skin barrier / wafer is part of a “flange” unit and the pouches include a closing ring that attaches mechanically to a mating piece on the flange. A common connection mechanism consists of a pressure fit snap ring.

In either one-or two-piece systems, having a properly-sized skin barrier is essential to the working of the pouching system. FIG. 1 shows conventional practices for changing an ostomy pouch, as advocated by medical and ostomy associations, such as the United Ostomy Associations of America, Inc. As shown, a user is generally required to measure their stoma and cut a skin barrier template every time the pouch is changed. Such practices often lead to poor skin barrier sizing and fit, which can be detrimental to proper functioning of the ostomy pouch system. Over time, weight gain or loss, pregnancy, hernias, ageing and other physical changes can also affect the size, and hence, fitting of a stoma as well.

Prior art systems have been developed in an attempt to ameliorate the field of fitting skin barriers for stoma patients. For example, WO 2011/123018 A1 describes a method for manufacturing body attachment wafers for ostomy devices from an image translated into a cutting geometry by means of a laser or water cutter. This system directly produces the skin barrier for use by the stoma patient, but does not account for ensuring that the stoma image is accurate unless produced by medical personnel, requiring a visit to a medical facility.

Similarly, EP 1275357 A2, US 2014/0236335 A1, AND KR 101937997 B1 describe similar methods and systems for fabricating customised ostomy devices, that typically require medical imaging and generally involve customised fabrication of stomas and pouching system connections. However, none of these prior art systems allow for a simple means whereby an ostomate is able to facilitate production of a custom ostomy skin barrier without the assistance of medical personnel using conventional sheets of ostomy skin barrier material.

In light hereof, Applicant has identified a need for an improved way to facilitate ostomy skin barrier fit and production from conventional materials and the current invention was conceived with this goal in mind.

### SUMMARY OF THE INVENTION

According to a first aspect of the invention there is provided a method for producing a custom ostomy skin barrier, said method comprising the steps of:
providing a scanner whereby a user is able to scan a torsal peristomal area, said scanner configured to generate a data image of the scanned peristomal area;
transmitting, by the scanner, the data image to a skin barrier production system; and
analysing, by the production system, the received data image in order to produce a skin barrier template whereby a custom ostomy skin barrier corresponding with a stoma in the scanned peristomal area is producible.

In an embodiment, the step of providing the scanner comprises providing or/and making use of a downloadable software application which is configured to adapt a user's mobile device, such as a mobile telephone or tablet, to scan the peristomal area and to generate and transmit said data image.

In an embodiment, the step of providing the scanner comprises providing a dedicated scanner device for scanning the peristomal area and generating and transmitting said data image.

In an embodiment, the data image comprises a depth map indicative of planar and depth data of the peristomal area to enable skin barrier template production accounting for planar dimensions and contours found in the peristomal area.

In an embodiment, the step of scanning comprises scanning the peristomal area together with a reference token, such as a coin of known dimensions, to provide a scale of dimension and/or parallax as part of the depth image.

In an embodiment, the scanner comprises first and second cameras for taking separate and offset images of the peristomal area to allow for parallax correction and/or compilation of the depth map.

In an embodiment, the scanner is configured to account for an angle between an optical axis of the first camera and an optical axis of the second camera to allow for parallax correction and/or compilation of the depth map.

In an embodiment, the scanner comprises a range finder to provide a distance from the scanner to the peristomal area during scanning to allow for parallax correction and/or compilation of the depth map.

In an embodiment, the scanner comprises an accelerometer, such as an inertial measurement unit (IMU), for capturing orientation data as part of generating the data image.

In an embodiment, the step of transmitting the data image comprises transmitting via the Internet and/or suitable telecommunications network.

In an embodiment, the step of analysing the data image by the skin barrier production system comprises analysing relative dimensions between a scanned reference token, such as a coin of known dimensions, to determine a desired dimension and/or parallax of the skin barrier template.

In an embodiment, the step of analysing the data image by the skin barrier production system comprises comparing pixel positions of a stoma (or other landmark pixels) between at least two data images and, if it is evident that there is parallax between the images, to apply computer vision depth perception techniques to allow for parallax correction and/or compilation of the depth map.

In an embodiment, the step of analysing the data image by the skin barrier production system comprises analysing orientation data in the data image to allow for parallax correction and/or compilation of the depth map.

In an embodiment, the method includes the step of producing the skin barrier template.

In an embodiment, the skin barrier template comprises a skin barrier configured for use with one-piece or two-piece ostomy pouch system.

In an embodiment, the skin barrier is manufactured from a hydrocolloid material.

In an embodiment, the skin barrier template comprises a template via which a user is able to self-produce a skin barrier for use with one-piece or two-piece ostomy pouch system, such as a stencil.

In an embodiment, the skin barrier template comprises a device via which a user is able to self-produce a skin barrier for use with one-piece or two-piece ostomy pouch system, such as a punch device, a cutter, or the like.

In an embodiment, the method includes the step of sending the produced skin barrier template to the user.

According to a second aspect of the invention there is provided a system for producing a custom ostomy skin barrier, the system comprising:
a scanner whereby a user is able to scan a torsal peristomal area, said scanner configured to generate a data image of the scanned peristomal area and to transmit the data image to a skin barrier production system; and
a skin barrier production system which is configured to receive and analyse the data image in order to produce a skin barrier template whereby a custom ostomy skin barrier corresponding with a stoma in the scanned peristomal area is producible.

In an embodiment, the scanner comprises a mobile device of a user, such as a mobile telephone or tablet, which is configured by means of a downloadable software application to adapt said mobile device for scanning the peristomal area and to generate and transmit said data image.

In an embodiment, the scanner comprises a dedicated scanner device for scanning the peristomal area and generating and transmitting said data image.

In an embodiment, the scanner scans the peristomal area together with a reference token, such as a coin of known dimensions, to provide a scale of dimension and/or parallax as part of the depth image.

In an embodiment, the data image comprises a depth map indicative of planar and depth data of the peristomal area to enable skin barrier template production accounting for planar dimensions and contours found in the peristomal area.

In an embodiment, the scanner comprises first and second cameras for taking separate and offset images of the peristomal area to allow for parallax correction and/or compilation of the depth map.

In an embodiment, the scanner is configured to account for an angle between an optical axis of the first camera and an optical axis of the second camera to allow for parallax correction and/or compilation of the depth map.

In an embodiment, the scanner comprises a range finder to provide a distance from the scanner to the peristomal area during scanning to allow for parallax correction and/or compilation of the depth map.

In an embodiment, the scanner comprises an accelerometer, such as an inertial measurement unit (IMU), for capturing orientation data as part of generating the data image.

In an embodiment, the scanner transmits the data image via the Internet and/or suitable telecommunications network.

In an embodiment, the skin barrier production system is configured to analyse the data image by analysing relative dimensions between a scanned reference token, such as a coin of known dimensions, to determine a desired dimension and/or parallax of the skin barrier template.

In an embodiment, the skin barrier production system is configured to analyse the data image by comparing pixel positions of a stoma (or other landmark pixels) between at least two data images and, if it is evident that there is parallax between the images, to apply computer vision depth perception techniques to allow for parallax correction and/or compilation of the depth map.

In an embodiment, the skin barrier production system is configured to analyse the data image by analysing orientation data in the data image to allow for parallax correction and/or compilation of the depth map.

In an embodiment, the skin barrier production system is configured to produce the skin barrier template.

In an embodiment, the skin barrier template comprises a skin barrier configured for use with one-piece or two-piece ostomy pouch system.

In an embodiment, the skin barrier is manufactured from a hydrocolloid material.

In an embodiment, the skin barrier template comprises a template via which a user is able to self-produce a skin barrier for use with one-piece or two-piece ostomy pouch system, such as a stencil.

In an embodiment, the skin barrier template comprises a device via which a user is able to self-produce a skin barrier for use with one-piece or two-piece ostomy pouch system, such as a punch device, a cutter, or the like.

According to a further aspect of the invention there is provided a method for producing a custom ostomy skin barrier, said method comprising the steps of:
providing a downloadable software application which is configured to adapt a user's mobile device, such as a mobile telephone or tablet, as a scanner to scan a torsal peristomal area, said scanner thereby further configured to generate a data image of the scanned peristomal area;
transmitting, by the mobile device, the data image to a skin barrier template production system;
analysing, by said template production system, the received data image in order to produce a skin barrier template whereby a custom ostomy skin barrier corresponding with a stoma in the scanned peristomal area is self-producible by the user from a conventional sheet of skin barrier material; and
sending the produced skin barrier template to the user to enable such self-production of an ostomy skin barrier.

According to a further aspect of the invention there is provided system for producing a custom ostomy skin barrier, the system comprising:
a downloadable software application which is configured to adapt a user's mobile device, such as a mobile telephone or tablet, as a scanner to scan a torsal peristomal area, said scanner thereby further configured to generate a data image of the scanned peristomal area; and
a skin barrier production system which is configured to:
   a. receive and analyse the data image in order to produce a skin barrier template whereby a custom ostomy skin barrier corresponding with a stoma in the scanned peristomal area is self-producible by the user from a conventional sheet of skin barrier material; and
   b. send the produced skin barrier template to the user to enable such self-production of an ostomy skin barrier.

According to a further aspect of the invention there is provided methods and associated systems for producing a custom ostomy skin barrier, substantially as herein described and/or illustrated.

### BRIEF DESCRIPTION OF THE DRAWINGS

The description will be made with reference to the accompanying drawings in which:

FIG. 1 is a diagrammatic representation of conventional prior art steps for fitting a skin barrier for an ostomy pouch system;

FIG. 2 is a diagrammatic representation of conventional prior art ostomy pouch systems, showing one-piece and two-piece systems;

FIG. 3 is diagrammatic representation of constituent parts of typical one-piece and two-piece ostomy pouch systems;

FIG. 4 Is a Diagrammatic: Representation of One embodiment of a system for producing a custom ostomy skin barrier, in accordance with aspects of the present invention; and

FIG. 5 is a diagrammatic representation of method steps, as represented by step blocks, for producing a custom ostomy skin barrier, in accordance with aspects of the present invention.

### DETAILED DESCRIPTION OF EMBODIMENTS

Further features of the present invention are more fully described in the following description of several non-limiting embodiments thereof. This description is included solely for the purposes of exemplifying the present invention to the skilled addressee. It should not be understood as a restriction on the broad summary, disclosure or description of the invention as set out above.

In the figures, incorporated to illustrate features of the example embodiment or embodiments, like reference numerals are used to identify like parts throughout. Additionally, features, mechanisms and aspects well-known and understood in the art will not be described in detail, as such features, mechanisms and aspects will be within the understanding of the skilled addressee.

Additionally, the accompanying figures do not represent engineering or design drawings, but provide a functional overview of the invention only. As a result, features and practical construction details required for various embodiments may not be indicated in each figure, but such construction requirements will be within the understanding of the skilled addressee.

Broadly, the present invention provides for a system 10 and associated method 30 for producing a custom ostomy skin barrier 12 for use with conventional ostomy pouch systems 14. In particular, the invention has been devised to improve fit of skin barriers used with such ostomy pouch systems 14.

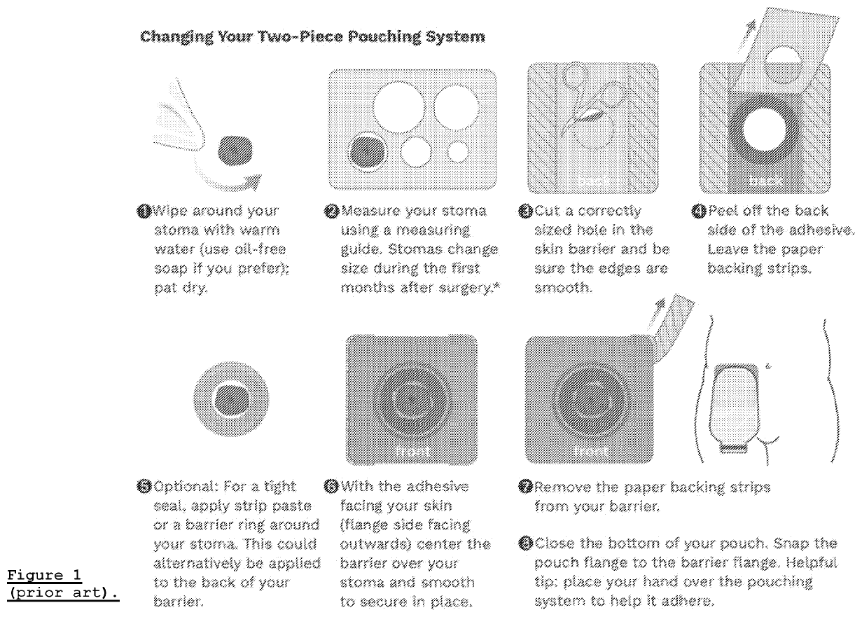

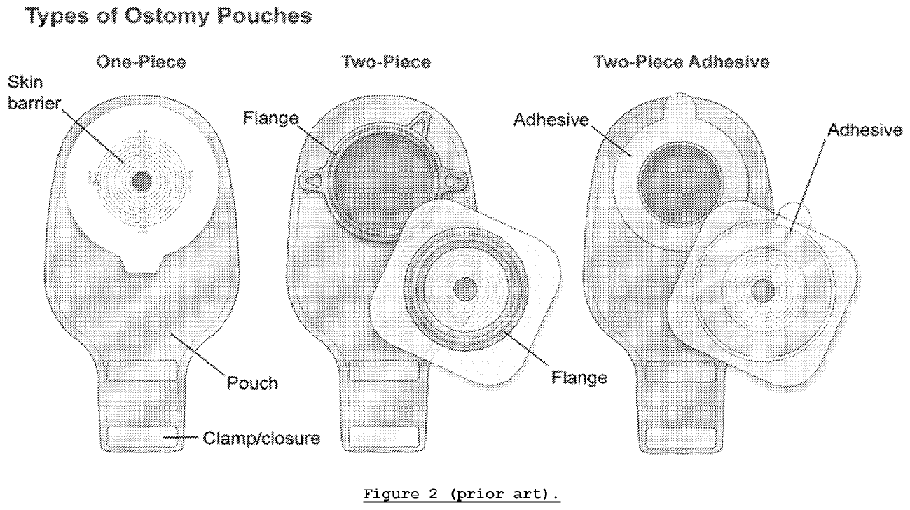

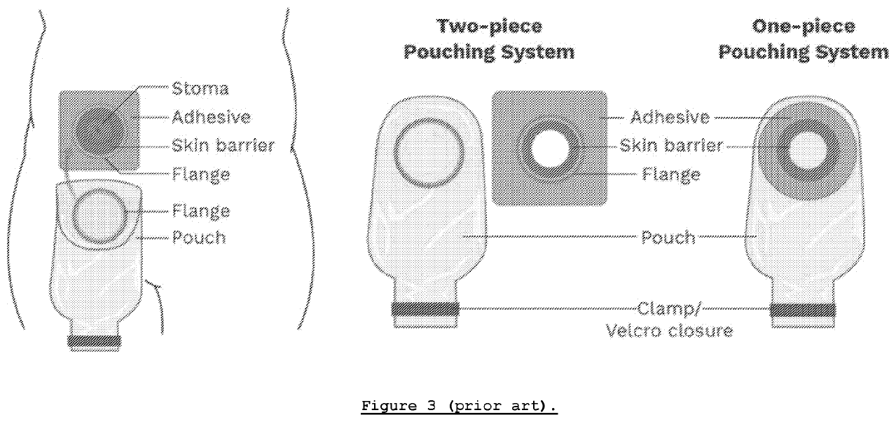

Conventionally, as shown in FIGS. 1 to 3, ostomy pouch systems require a user to measure and cut their own skin barriers each time the pouch is changed. Typically, this involves measuring a stoma against a rough measuring guide, as shown in FIG. 1, and then manually cutting a general skin barrier template with circular diameters indicated thereon (shown in FIG. 2), as per the measuring guide.

Of course, stomas are rarely perfectly circular and the better the skin barrier fits over and about the stoma, the better the ostomy pouch system works and the more positive the user experience. Additionally, stomas can change size and/or shape over time, making such skin barrier fitment a dynamic and ongoing process, typically required on a daily or similarly frequent basis.

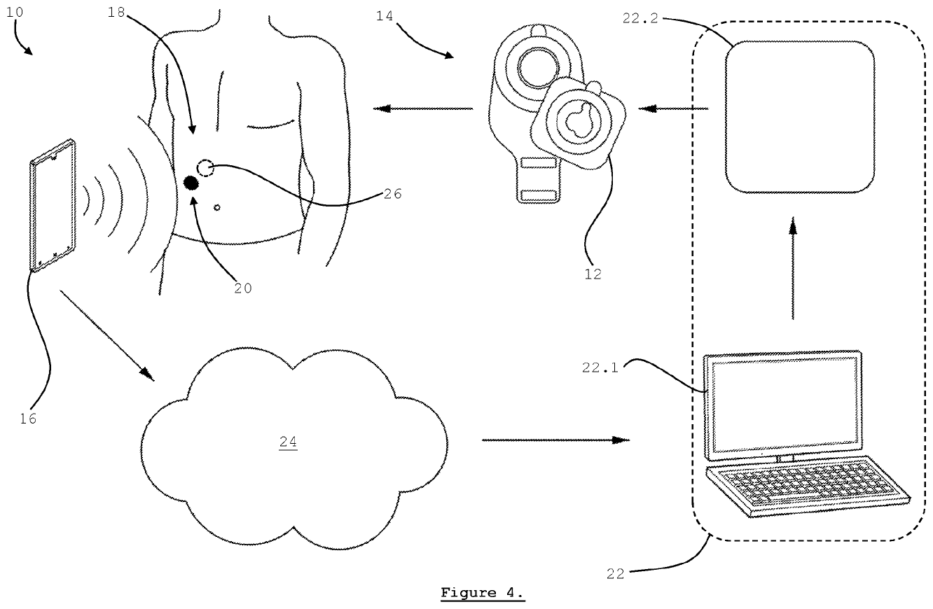

Referring now to FIG. 4. of the accompanying drawings, there is broadly exemplified a system 10 for producing a custom ostomy skin barrier 12 for use with a conventional ostomy pouch system 14. The system 10 generally comprises a scanner 16 and a skin barrier production system 22 for producing skin barrier templates, as described in more details below. In the example, the skin barrier production system 22 comprises a computer system 22.1 and a skin barrier template manufacturer 22.2, but variations hereon are possible and anticipated. For example, the production computer system 22.1 and the template manufacturer or manufacturing facility 22.2 may be separate, combined, or the like.

The scanner 16 is provided whereby a user is able to scan a torsal peristomal area 18, the scanner 16 configured to generate a data image of the scanned peristomal area 18 and to transmit the data image to the skin barrier production system 22. In one embodiment, the scanner 16 comprises a mobile device of a user, such as a mobile telephone or tablet, which is configured by means of a downloadable software application or ‘app’ to adapt the mobile device for scanning the peristomal area 18 and to generate and transmit the data image. For example, in one embodiment an existing or conventional camera app of a mobile device may be used to scan the peristomal area. In another embodiment, a dedicated app may be provided for scanning via a camera, or the like. Alternatively, or additionally, the scanner 16 may also comprises a dedicated scanner device for scanning the peristomal area 18 and generating and transmitting said data image. For example, a dedicated medical device for use by a physician or technician.

In one embodiment, the user scans the peristomal area 18 together with a reference token 26, such as a coin of known dimensions, to provide a scale of dimension and/or parallax as part of the depth image, e.g. the reference token 26 is placed proximate the stoma during scanning. In such an example, the skin barrier production system 22 is typically configured to analyse the data image by analysing relative dimensions between such a scanned reference token 26, such as a coin of known dimensions, to determine a desired dimension of the skin barrier template.

For example, in one embodiment, the app which adapts a user's mobile phone to form the scanner 16 may be configured to instruct and/or require the user to place a suitable reference token 26 of known dimensions proximate the peristomal area 18, such as indicating a virtual window or box on a camera frame within which the reference token is to be placed, or the like. The app may also be configured to facilitate printing of the reference token 26 by the user for inclusion as part of the scanning process, or the like, e.g. an optical recognition pattern of known and predetermined size whereby relative dimensions is obtainable after scanning to allow for parallax correction and/or compilation of a depth map, or the like.

In a typical embodiment, the data image generated by the scanner comprises a depth map indicative of planar and depth data of the peristomal area 18 to enable skin barrier template production accounting for planar dimensions and contours found in the peristomal area 18, i.e. contours, height differences, etc.

In one embodiment, the scanner 16 may comprise first and second cameras for taking separate and offset images of the peristomal area 18 to allow for parallax correction and/or compilation of the depth map. For example, modern mobile phones often include more than one camera, which can be used in order to scan and generate a depth map. In such an embodiment, the scanner 16 may be configured to account for an angle between an optical axis of the first camera and an optical axis of the second camera to allow for parallax correction and/or compilation of the depth map.

In another embodiment, the scanner 16 may comprise a range finder, such as a lidar device, to provide a distance from the scanner 16 to the peristomal area 18 during scanning to allow for parallax correction and/or compilation of the depth map. In one embodiment, the scanner 16 may also comprises an accelerometer, such as an inertial measurement unit (IMU), for capturing orientation data as part of generating the data image during scanning. Such orientation data may be used in parallax correction and/or compilation of the depth map.

In an embodiment, the scanner 16 typically transmits the data image via the Internet and/or suitable telecommunications network 24. The skin barrier production system 22 is generally configured to receive and analyse the data image from the scanner 16 in order to produce a skin barrier template whereby the custom ostomy skin barrier 12 corresponding with the stoma 20 in the scanned peristomal area 18 is producible.

In one embodiment, the skin barrier production system 20 is configured to analyse the data image by comparing pixel positions of a stoma (or other landmark pixels, such as skin marks or blemishes, the umbilicus, or the like) between at least two data images and, if it is evident that there is parallax between the images, to apply computer vision depth perception techniques to allow for parallax correction and/or compilation of the depth map. Such techniques are within the understanding of the skilled addressee and will not be described in detail herein.

In one embodiment where the scanner provides orientation data, the skin barrier production system is typically configured to analyse the data image by analysing such orientation data in the data image to allow for parallax correction and/or compilation of the depth map.

In one embodiment, the skin barrier production system 22 is configured to produce the skin barrier template. This skin barrier template may broadly comprise a skin barrier 12, a stencil or the like whereby a user is able to self-produce a skin barrier from a conventional sheet of skin barrier material for use with one-piece or two-piece ostomy pouch system, or the template may comprise a device via which a user is able to self-produce a skin barrier, such as a punch device, a cutter, or the like.

For example, the skin barrier production system 22 may produce a skin barrier 12 directly, typically from a hydrocolloid material, which includes an aperture therein which corresponds with the scanned stoma 20, or a template stencil which corresponds with the scanned stoma 20. Such a stencil may allow a user to trace and cut an aperture in a conventional sheet of skin barrier material which corresponds with the scanned stoma 20. In a yet further embodiment, the skin barrier production system 22 may produce a device, such as a hole punch, which can be used to punch an aperture in a conventional skin barrier sheet of material, the aperture precisely matched to the scanned stoma. In a further example, the skin barrier template may also include an electronic file printable by an end user so that such printout represents the template for producing the custom ostomy skin barrier via cutting it from a conventional sheet of skin barrier material.

In this manner, a skin barrier 12 can be produced with an aperture therein which closely matches the scanned stoma, thereby enabling a closer and more accurate fitting skin barrier.

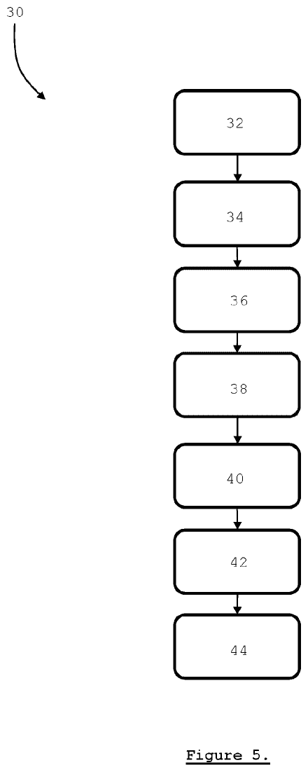

With reference now to FIG. 5 of the accompanying drawings, the skilled addressee is to appreciate that the present invention also provides for an associated method 30 for producing a custom ostomy skin barrier 12. Such a method 30 broadly comprises the steps of providing 32 a scanner 16 whereby a user is able to scan a torsal peristomal area 18, said scanner 16 configured to generate 34 a data image of the scanned peristomal area 18, and transmitting 36, by the scanner 16, the data image to a skin barrier production system 22. The method 30 also typically includes the step of analysing 38, by the production system 22, the received data image in order to produce 40 a skin barrier template whereby a custom ostomy skin barrier 12 corresponding with a stoma in the scanned peristomal area is producible. In one embodiment, the method 30 may also include the step of producing 42 the skin barrier 12, as well as sending 44 the produced skin barrier template to the user.

For example, the method 30 may facilitate an on-demand and/or prescription service for providing custom ostomy skin barriers. Such a service may comprise users being able to scan their stomas in order to receive custom-made to order skin barriers 12 and/or templates that has been sized, shaped and/or dimensioned specifically to suit their stomas and peristomal areas to facilitate secure fit and seal of an ostomy pouch system. The invention may facilitate the production of custom skin barrier as part of one-piece or two-piece ostomy pouch systems, as required. Advantageously, due to stomas varying in shape and/or size over time, the present invention allows a user to obtain an up-to-date and fitted skin barrier and/or template on demand.

In the example embodiments, well-known processes, well-known device structures, and well-known technologies are not described in detail, as such will be readily understood by the skilled addressee. Optional embodiments of the present invention may also be said to broadly consist in the parts, elements and features referred to or indicated herein, individually or collectively, in any or all combinations of two or more of the parts, elements or features. Where specific integers are mentioned herein which have known equivalents in the art to which the invention relates, such known equivalents are deemed to be incorporated herein as if individually set forth.

It is to be appreciated that reference to “one example” or “an example” of the invention, or similar exemplary language (e.g., “such as”) herein, is not made in an exclusive sense. Various substantially and specifically practical and useful exemplary embodiments of the claimed subject matter are described herein, textually and/or graphically, for carrying out the claimed subject matter. Accordingly, one example may exemplify certain aspects of the invention, whilst other aspects are exemplified in a different example. These examples are intended to assist the skilled person in performing the invention and are not intended to limit the overall scope of the invention in any way unless the context clearly indicates otherwise.

Variations (e.g. modifications and/or enhancements) of one or more embodiments described herein might become apparent to those of ordinary skill in the art upon reading this application. The inventor(s) expects skilled artisans to employ such variations as appropriate, and the inventor(s) intends for the claimed subject matter to be practiced other than as specifically described herein.

The use of the terms “a”, “an”, “said”, “the”, and/or similar referents in the context of describing various embodiments (especially in the context of the claimed subject matter) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. As used herein, the term “and/or” includes any and all combinations of one or more of the associated listed items. No language in the specification should be construed as indicating any non-claimed subject matter as essential to the practice of the claimed subject matter.

Any method steps, processes, and operations described herein are not to be construed as necessarily requiring their performance in the particular order discussed or illustrated, unless specifically identified as an order of performance. It is also to be understood that additional or alternative steps may be employed.

1. A method for producing a custom ostomy skin barrier, said method comprising the steps of:
providing a downloadable software application which is configured to adapt a user's mobile device, such as a mobile telephone or tablet, as a scanner to scan a torsal peristomal area together with a reference token, such as a coin of known dimensions, to provide a scale of dimension and/or parallax of the scanned area, said scanner thereby further configured to generate a data image comprising such scale of dimension and/or parallax of the scanned peristomal area;
transmitting, by the mobile device, the data image to a skin barrier template production system;
analysing, by said template production system, the received data image to identify relative dimensions between the scanned reference token to determine a desired dimension and/or parallax of the scanned peristomal area in order to produce a skin barrier template whereby a custom ostomy skin barrier corresponding with a stoma in the scanned peristomal area is self-producible by the user from a conventional sheet of skin barrier material; and
sending the produced skin barrier template to the user to enable such self-production of an ostomy skin barrier.

2. The method of claim 1, wherein the step of producing the skin barrier template by the template production system comprises producing a template selectable from a group consisting of a stencil whereby a user is enabled to trace and cut an aperture in a conventional sheet of skin barrier material, and a cutting device which is configured to cut a custom skin barrier from a conventional sheet of skin barrier material.

3. The method of claim 1, wherein the step of producing the skin barrier template by the template production system comprises producing an electronic file printable by the end user so that such printout provides a stencil for producing the custom ostomy skin barrier by tracing and cutting an aperture in a conventional sheet of skin barrier material.

4. The method of claim 1, wherein the step of providing the downloadable software application which is configured to adapt a user's mobile device as a scanner includes said application configuring first and second cameras of said device to capture separate and offset images of the peristomal area to allow for parallax correction and/or compilation of a depth map indicative of planar and depth data of the peristomal area to enable skin barrier template production accounting for planar dimensions and contours found in the peristomal area.

5. The method of claim 4, wherein the scanner is configured to account for an angle between an optical axis of the first camera and an optical axis of the second camera to allow for parallax correction and/or compilation of the depth map.

6. The method of claim 5, wherein the scanner comprises a range finder to provide a distance from the scanner to the peristomal area during scanning to allow for parallax correction and/or compilation of the depth map.

7. The method of claim 1, wherein the step of analysing the data image by the skin barrier production system comprises comparing pixel positions of a stoma, the reference token, and/ or other landmark pixels between at least two data images and, if it is evident that there is parallax between the images, to apply computer vision depth perception techniques to allow for parallax correction and/or compilation of a depth map indicative of planar and depth data of the peristomal area.

8. The method of claim 1, wherein the scanner comprises an accelerometer, such as an inertial measurement unit (IMU), for capturing orientation data as part of generating the data image, and wherein the step of analysing the data image by the skin barrier production system comprises analysing such orientation data in the data image to allow for parallax correction and/or compilation of a depth map indicative of planar and depth data of the peristomal area.

9. A system for producing a custom ostomy skin barrier, the system comprising:
a downloadable software application which is configured to adapt a user's mobile device, such as a mobile telephone or tablet, as a scanner to scan a torsal peristomal area together with a reference token to provide a scale of dimension and/or parallax of the scanned area, said scanner thereby further configured to generate a data image of the scanned peristomal area; and
a skin barrier production system which is configured to:
a. receive and analyse the data image to identify relative dimensions between the scanned reference token to determine a desired dimension and/or parallax of the scanned peristomal area in order to produce a skin barrier template whereby a custom ostomy skin barrier corresponding with a stoma in the scanned peristomal area is self-producible by the user from a conventional sheet of skin barrier material; and
b. send the produced skin barrier template to the user to enable such self-production of an ostomy skin barrier.

10. The system of claims 9, wherein the skin barrier template produced by the template production system comprises a template selectable from a group consisting of a stencil whereby a user is enabled to trace and cut an aperture in a conventional sheet of skin barrier material, a cutting device which is configured to cut a custom skin barrier from a conventional sheet of skin barrier material, and an electronic file printable by the end user so that such printout provides a stencil for producing the custom ostomy skin barrier by tracing and cutting an aperture in a conventional sheet of skin barrier material.

11. The system of claim 9, wherein the application which adapts the mobile device as a scanner is configured to configure first and second cameras of said device to capture separate and offset images of the peristomal area to allow for parallax correction and/or compilation of a depth map indicative of planar and depth data of the peristomal area to enable skin barrier template production accounting for planar dimensions and contours found in the peristomal area.

12. The system of claim 11, wherein the scanner is configured to account for an angle between an optical axis of the first camera and an optical axis of the second camera to allow for parallax correction and/or compilation of the depth map, said skin barrier production system further configured to analyse the data image by comparing pixel positions of a stoma, the reference token, and/or other landmark pixels between at least two data images and, if it is evident that there is parallax between the images, to apply computer vision depth perception techniques to allow for parallax correction and/or compilation of the depth map.

13. The system of claim 9, wherein the scanner comprises a range finder to provide a distance from the scanner to the peristomal area during scanning to allow for parallax correction and/or compilation of a depth map indicative of planar and depth data of the peristomal area to enable skin barrier template production accounting for planar dimensions and contours found in the peristomal area.

14. The system of claim 9, wherein the scanner comprises an accelerometer, such as an inertial measurement unit (IMU), for capturing orientation data as part of generating the data image, and wherein the skin barrier production system is configured to analyse the data image by analysing orientation data in the data image to allow for parallax correction and/or compilation of a depth map indicative of planar and depth data of the peristomal area to enable skin barrier template production accounting for planar dimensions and contours found in the peristomal area.
